(19) United States
(12) Patent Application Publication
(10) Pub. No.: US 2011/0170068 A1
(43) Pub. Date: Jul. 14, 2011

(54) Method and apparatus for self-examination of the eye

(75) Inventor: Ilan Dan-Gur, New Westminster (CA)

(73) Assignee: Opticom Data Research

(21) Appl. No.: 12/928,071

(22) Filed: Dec. 3, 2010

(51) Int. Cl. A61B 3/10

(52) U.S. Cl. 351/214; 351/246

(57) ABSTRACT

A method to enable the use of hand-held mobile devices for self-examination of opacities in the eye. Specifically, an image of a pinhole light source is created on the screen of a mobile device. The user then holds the screen of the mobile device close to the eye such that the pinhole image is along the optical axis of the eye. A detailed image of opacities in the eye is thus formed on the retina. The following features are used for optimizing the image of opacities: The light intensity of the pinhole image is adjustable. The pinhole image can be pulsed. The location of the pinhole image on the screen can be changed.

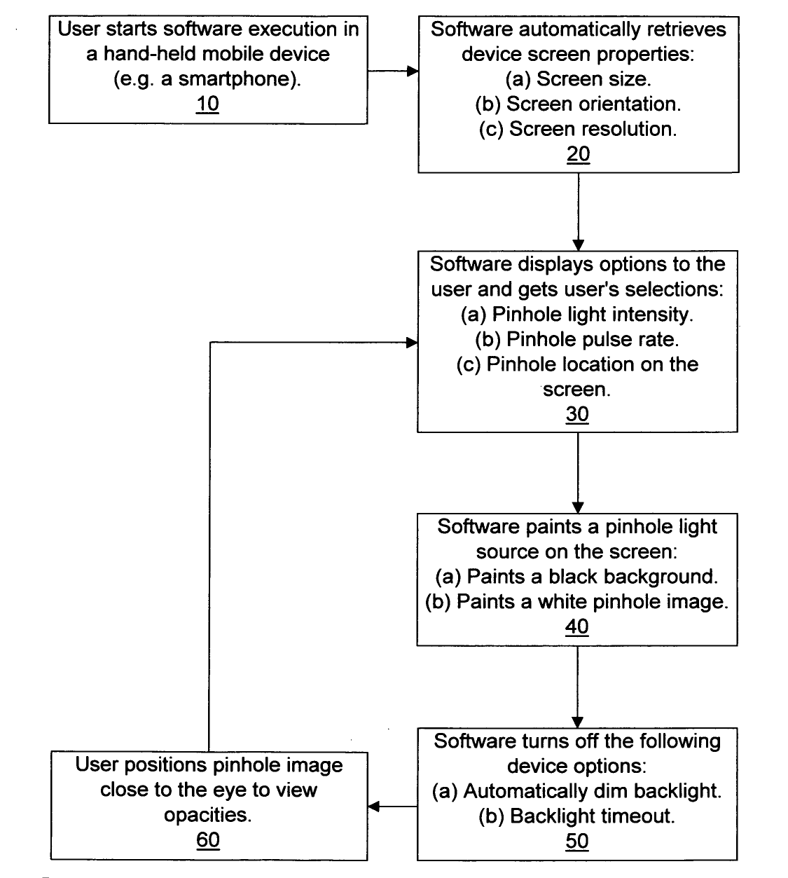

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to ophthalmic methods and apparatus, and more particularly to methods of using hand-held mobile devices for the purpose of self-examination of the eye.

2. Discussion of Prior Art

Health related devices for self-testing at home are employed by an increasing number of individuals to monitor health conditions. With respect to ocular health, there are various conditions that manifest themselves as opacities in the eye that an individual may want to monitor. Such conditions include floaters, cataracts, lesions, scar tissue, retinal burns, as well as debris and scratches in contact lenses while wearing them. In addition, an individual may want to test the functioning of the iris, i.e. the change in pupil size as a function of the amount of light entering the eye. Ophthalmic devices to monitor such conditions must be safe to use. Preferably, they should also be easy to operate, have a small size and be affordable.

It is well known that when placing a pinhole light source close to the eye, opacities in the eye become highly visible. In essence, a beam of light illuminates part of the retina, with every such point on the retina, in theory, illuminated by a single ray of light. Sharply defined shadows of opacities in the eye are thus created on the retina and are easily viewable by the user. The shadows appear on the retina within a circle of light, the edge of the circle itself being a shadow of the edge of the pupil of the eye.

When the pinhole light source is positioned at the anterior focus of the eye (about 16 millimeters from the surface of the eye), the eye collimates the light into a parallel beam inside the eye. As the pinhole light source is moved closer to the eye, up to the surface of the eye, the beam inside the eye becomes increasingly divergent and two advantages become noticeable. First, a larger volume of the eye is imaged on a larger part of the retina, and second, the size of some shadows on the retina is magnified (depending on the location of the opacities within the eye).

U.S. Pat. No. 3,903,870 to Berndt (1975) shows a technique and an apparatus for self-examination of the eye. The technique uses a point-source of light placed at the anterior focus of the eye, creating a parallel beam of light within the eye which enables viewers to see and inspect their own visual system. In his preferred embodiment, Berndt uses the output of an optical fiber to create a point-source of light. Berndt describes other possible embodiments of his invention, including using a pinhole instead of an optical fiber.

U.S. Pat. No. 4,682,867 to Gould (1987), and U.S. Pat. No. 4,902,124 to Roy, Sr. et al. (1990), show methods and means for self-examination of the eye. A pinhole light source is created using a light source, a diffuser and a pinhole, the diffuser creating a homogeneous radiation at the pinhole. When the pinhole is positioned along the viewer's optical axis and close to the eye, moving and fixed opacities in the eye are imaged on the retina of the viewer.

All the above-mentioned prior art suffer from a number of disadvantages:

(a) A specialized apparatus is used, which is costly to manufacture. The user then needs to buy this apparatus specifically for the purpose of monitoring the eye.

(b) They offer no inherent way for the user to adjust the intensity of the pinhole light source, nor do they discuss the need to adjust the intensity. Being able to adjust the intensity can be useful since people have varied sensitivities to light, intensity.

(c) They offer no inherent way to automatically pulse the pinhole light source on and off, nor do they discuss the need to pulse it. Pulsing the pinhole light source on and off can accentuate the appearance of floaters as they move around inside the eye and change position. The pulse rate should be, for example, 0.5 seconds “on” and 0.5 seconds “off,” and without the user needing to click a switch, so that the location of the pinhole light source remains stable with respect to the eye.

(d) They use a light source which is designed for illumination rather than for direct viewing, and may not be safe to use close to the eye due to UV and infrared radiation which typically exist in such light sources.

### Objects and Advantages

Accordingly, several objects and advantages of the present invention are:

(a) to provide a method for using hand-held mobile devices for self-viewing opacities in the eye. Such devices are already owned and used by many people and a user would only need to install a software or view a Web page in order to perform the ocular self-exam. No specialized hardware would be required.

(b) to provide an option to adjust the intensity of the pinhole light source to the sensitivity level of the user.

(c) to provide an option to automatically pulse the pinhole light source on and off.

(d) to use the screen of hand-held mobile devices as the light source for the self-examination. Such screens are inherently designed for direct viewing.

Other objects and advantages of the present invention will become apparent from a consideration of the ensuing description and drawing.

### DRAWINGS

The sole FIGURE is a flowchart of an example embodiment of the present invention.

### DETAILED DESCRIPTION OF THE PREFERRED EMBODIMENT

The present invention makes use of hand-held mobile devices, such as cell phones, smartphones, and personal digital assistants (PDAs), for self-examination of the eye.

Throughout the disclosure, the terms “hand-held mobile device,” “mobile device” and “device” are used interchangeably. Also, the terms “pinhole light source,” “pinhole image” and “pinhole” are used interchangeably. In hand-held mobile devices, “screen brightness” is sometimes called “backlight brightness.”

In the preferred embodiment, an image depicting a white pinhole light source surrounded by a black background is created on the screen, or display, of the mobile device. The user then positions the pinhole image close to the eye to view opacities.

The process of the preferred embodiment includes six steps as shown in the sole FIGURE:

1. The user starts the software execution in the hand-held mobile device (block 10).

2. The software automatically retrieves from the device the screen properties (block 20):
(a) Screen size (e.g. in units of pixels). The screen size data is used to paint, or draw, the pinhole image at specific locations on the screen.
(b) Screen orientation (e.g. landscape or portrait). Some devices have sensors that can detect the orientation of the screen with respect to the user. In such devices, the orientation data is used, along with the screen size data, to paint the pinhole image at specific locations on the screen.
(c) Screen resolution (e.g. in units of pixels per inch). The screen resolution data is used to calculate the size of the pinhole image on the screen (e.g. 100×100 micrometers).

3. The software displays options to the user and gets the user's selections (block 30):
(a) Pinhole light intensity. The user selects a pinhole light intensity, for example between ten arbitrary levels of 1-10, and the software informs the user of the corresponding pinhole size that will be used to achieve this intensity, for example 0.4 millimeters. The software controls the intensity of the pinhole light source by changing the screen brightness and the number of pixels that are part of the pinhole image. At the lower intensity levels (e.g. levels 1-3), the software uses a single pixel to depict the pinhole light source, and the software controls the light intensity by changing the screen brightness of the hand-held mobile device. At the higher intensity levels (e.g. levels 4-10), the software sets the device screen brightness to 100%, and uses more pixels to form the pinhole image. Ideally, the pinhole image size should be a single pixel so that it is as close as possible to a point-source. In current hand-held mobile devices the size of a single pixel is about 70×70 micrometers and up to about 150×150 micrometers. However, depending on the particular mobile device, the light sensitivity of the user, and the type of opacities, a single pixel may not provide enough light intensity to see the details of the shadows on the retina, even if the screen brightness is set to 100% in the mobile device. In that case the software can increase the intensity by using a few white pixels together creating an area, or an image, of a square on the black background. This increases the size of the pinhole light source and may contribute to reducing the resolution of the image on the retina, i.e. less details of the shadows will be visible. The optimal pinhole light intensity that should be used, therefore, is a balance between having enough light intensity while keeping the pinhole image size as small as possible. The user selects this optimal pinhole light intensity by experimenting with the various pinhole intensity levels until the best image on the retina is achieved.
(b) Pinhole pulse rate. The user decides whether the pinhole light source is steady (i.e. continuous) or pulsed on and off, and at what rate (e.g. 0.5 seconds “on” and 0.5 seconds “off”). Pulsing the pinhole light source on and off can improve the contrast on the retina between consecutive images of floaters that move inside the eye. In order to pulse the pinhole light source “off,” the software paints the pinhole image in the same color as the screen background color.
(c) Pinhole location on the screen. The user selects a pinhole location on the screen, for example “Center” or “Upper-Left Corner.” If the device has a touch screen, the software lets the user select the pinhole location by clicking on the screen at the desired location. Since the pinhole image should be aligned along the optical axis of the eye and as close as possible to the eye, the optimal location of the pinhole image on the screen depends on the specific hand-held mobile device size and shape, the screen orientation with respect to the user, and the facial features of the user.

4. The software paints a pinhole light source on the screen (block 40):
(a) The software paints the screen in black color as a background to the pinhole image. The black pixels, as well as the size of the device screen itself, reduce, or stop, any light other than that of the pinhole image itself from entering the eye, thus producing the effect of a pinhole light source.
(b) The software paints the pinhole image as per the user's selections (block 30) and screen properties (block 20).

5. The software turns off the following device options (block 50):
(a) Automatically dim backlight. Some devices have sensors that automatically dim the screen brightness according to the ambient light they detect, in order to reduce power consumption. However, during the eye exam, i.e. when the pinhole image is on the screen, it is preferable that the screen brightness remains constant; otherwise, the size of the pinhole image may need to change to compensate for the change in intensity.
(b) Backlight timeout. Some device screens automatically become blank when the device is not in use (e.g. when no button or key is pressed for a few seconds) in order to reduce power consumption. However, the pinhole image size is, at most, a few pixels, so the screen consumes very little power. The software prevents devices from automatically turning the screen off during the eye exam, i.e. when the pinhole image is on the screen.

6. The user positions the pinhole image close to the eye to view opacities (block 60). The user holds the screen of the mobile device close to the eye such that the white pinhole image is along the optical axis of the eye and as close as possible to the eye without touching the eye with the device screen. Shadows of opacities in the eye thus form a detailed image on the retina. Further, the user can move eyes up-down or left-right to induce movement of floaters inside the eyes, and then look into the pinhole light source to view images of the moving floaters. The closer the pinhole image is to the eye, the larger the area of the retina illuminated by the pinhole image. While a user may be able to see colored lines (usually red, green, and blue) that are radiated from subpixels in screens of mobile devices, these faint color lines do not interfere with the ability to view a clear and sharp image of opacities in the eye.

In addition to the above features, the size of the screen of hand-held mobile devices inherently provides protection against accidentally touching the eye surface during the self-examination since the screen will first touch the eyelashes or eyelids before touching the surface of the eye. Even if such a contact is made between the device screen and the surface of the eye, the smoothness of the screen glass is unlikely to damage the eye.

The software as described in the preferred embodiment can be implemented using various programming languages (e.g. Java, C#, C++, Objective C). The target hardware for running the software are hand-held mobile devices such as cell phones, smartphones, and PDAs. Such devices have a CPU, memory, screen, input capability (such as a keypad or touch screen), optionally a network connection, and a battery.

### CONCLUSION

### Alternative Embodiments, and Scope of Invention

A method according to the description above thus enables the use of hand-held mobile devices for self-monitoring opacities in the eye, such as floaters, and noticing changes over time. Further, the method makes it possible for users to change the intensity and pulse rate of the pinhole light source for optimizing the performance of the device for self-examination of the eye.

A number of alternative embodiments of the present invention are possible as is obvious to those skilled in the art. For example:

One alternative embodiment controls the intensity of the pinhole light source by changing the color of the pinhole image (instead of changing the screen brightness), as well as the number of pixels that are part of the pinhole image. With regard to changing the pinhole color, a white color would represent maximum intensity while black color would represent minimum intensity.

Another alternative embodiment lets the user directly select the pinhole size instead of selecting intensity levels of the pinhole light source. In this embodiment the pinhole image color may be white and the screen brightness may be set at 100%.

Another alternative embodiment increases the pinhole light intensity by adding white pixels along a line (rather than by creating an image of a square).

Another alternative embodiment asks the mobile device user to manually adjust certain settings of the device. Some devices do not allow third-party software that is installed on the device to adjust certain device settings such as “screen brightness” or “automatically dim backlight.” However, the device user can still change the settings manually. In such devices the software might ask the user, for example, to manually set the “screen brightness” setting to 100% and to turn off the “automatically dim backlight” setting.

Another alternative embodiment paints the pinhole image and black background in the Web browser of a mobile device. This embodiment can be implemented using a server-side script (e.g. ASP, PHP), client-side script (e.g. JavaScript), or a markup language (e.g. HTML).

While the present invention has been shown and described in the context of specific embodiments, it will be understood by those skilled in the art that numerous changes in the details may be made without departing from the scope and spirit of the invention.

Accordingly, the scope of the invention should be determined not by the embodiments illustrated, but by the appended claims and their legal equivalents.

What is claimed is:

1. A method of self-examination of opacities in the human eye, comprising:
(a) providing a hand-held mobile device having a screen,
(b) using said device to paint an image on said screen, said image comprising:
(1) a pinhole image consisting of an area smaller than about 2 square millimeters, said area consisting of pixels of mostly lighter colors,
(2) a background for said pinhole image, said background consisting of pixels of mostly darker colors,
(c) positioning said screen close to the eye such that said pinhole image is along the optical axis of the eye,
whereby opacities in the eye create clear and sharp shadows on the retina that are viewed by the user.

2. A method according to claim 1, wherein said pinhole image is white color and said background is black color.

3. A method according to claim 1, wherein said pinhole image has a shape selected from the group consisting of a square and a line.

4. A method according to claim 1, wherein said pinhole image is completely surrounded by said background.

5. A method according to claim 1, wherein said pinhole image is partly surrounded by said background and partly surrounded by the edge of said screen.

6. A method according to claim 1, further using said device to let the user select an intensity level for said pinhole image.

7. A method according to claim 6, wherein said intensity level being determined by adjusting at least one parameter selected from the group consisting of the size of said pinhole image, the colors of said pinhole image and the brightness of said screen.

8. A method according to claim 1, further using said device to let the user select a pulse rate for said pinhole image.

9. A method according to claim 1, further using said device to let the user select the location of said image on said screen.

10. A method according to claim 1, further preventing said device from automatically dimming said screen when said image is depicted on said screen.

11. A method according to claim 1, wherein the instructions for said device to paint said image on said screen are in HTML.

12. An apparatus for self-examination of opacities in the human eye, comprising:
(a) a hand-held mobile device having a screen,
(b) a software installed in said device, wherein said software, when executed by said device, causes said device to paint on said screen an image comprising:
(1) a pinhole image consisting of an area smaller than about 2 square millimeters, said area consisting of pixels of mostly lighter colors,
(2) a background for said pinhole image, said background consisting of pixels of mostly darker colors,
whereby opacities in the eye become conspicuous when said screen is positioned by the user close to the eye such that said pinhole image is along the optical axis of the eye.

13. An apparatus according to claim 12, wherein said pinhole image is white color and said background is black color.

14. An apparatus according to claim 12, wherein said pinhole image has a shape selected from the group consisting of a square and a line.

15. An apparatus according to claim 12, wherein said pinhole image is completely surrounded by said background.

16. An apparatus according to claim 12, wherein said pinhole image is partly surrounded by said background and partly surrounded by the edge of said screen.

17. An apparatus according to claim 12, wherein said software, when executed by said device, further causes said device to set the intensity level of said pinhole image according to a selection by the user.

18. An apparatus according to claim 17, wherein said intensity level being determined by adjusting at least one parameter selected from the group consisting of the size of said pinhole image, the colors of said pinhole image and the brightness of said screen.

19. An apparatus according to claim 12, wherein said software, when executed by said device, further causes said device to pulse said pinhole image at a rate determined by the user.

20. An apparatus according to claim 12, wherein said software, when executed by said device, further causes said device to paint said image on said screen at a location determined by the user.
